(19) United States
(12) Patent Application Publication
(10) Pub. No.: US 2009/0196925 A1
(43) Pub. Date: Aug. 6, 2009

(54) NATURAL PRODUCT COATING FOR TABLETS

(76) Inventors: Scott P. Brady, Seymour, WI (US); Robert C. Doster, Oneida, WI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 12/168,619

(22) Filed: Jul. 7, 2008

Related U.S. Application Data
(60) Provisional application No. 61/026,315, filed on Feb. 5, 2008.

(51) Int. Cl. A61K 9/36; A61K 47/36; A61K 47/06; A61K 9/30

(52) U.S. Cl. 424/479; 514/772; 514/777; 424/475

(57) ABSTRACT

An all-natural tablet coating composition comprised entirely of Certified Organic ingredients and organic compliant ingredients which can be applied to a compressed tablet core using conventional film coating techniques is provided. The coating is useful for preparing organic dietary supplements in a coated tablet form that exhibits good physical and chemical stability, good aesthetics, and which is free of synthetic materials.

### CROSS REFERENCE TO RELATED APPLICATIONS

This application claims, under 35 U.S.C. §119(e), the benefit of U.S. Provisional Application No. 61/026,315, filed Feb. 5, 2008, entitled “NATURAL PRODUCT COATING FOR TABLETS,” the entire disclosure of which is incorporated herein by reference.

### FIELD OF THE INVENTION

This invention relates to the field of nutritional supplementation, and more particularly to coatings for solid orally administered dietary supplements in a tablet form.

### BACKGROUND OF THE INVENTION

Coated tablets are well-known in the art and have been employed for over 1,000 years. For example, it is reported that the mucilage of psyllium seeds was used to coat pills having an offending taste at least as early as the 9^{th} century. Subsequently, it is reported that gold, silver, gelatin, and waxes have been employed as tablet coatings.

Unquestionably, a significant portion, if not most, pharmaceutical and/or nutraceutical solid dosage forms are provided with a coating that typically performs a plurality of functions. Tablet coatings are used for protecting the active ingredient in the tablet core from air, moisture and light to improve stability and extend useful shelf life of the tablet. Coatings are also used for masking unpleasant taste and odor; and for making the tablet easier to swallow. In addition, tablet coatings can be used to improve the appearance of the tablet, and to improve the mechanical integrity of the tablet by making it more resistant to abrasion, attrition and the like.

Coatings that are currently most commonly used include polymer film coatings and sugar coatings. Other types of coatings and coating techniques, such as compression coating in which dry coating materials are compacted onto a tablet core, are generally unpopular because of their mechanical complexity and resulting high cost.

While it is conceivable that a sugar coating could be used to coat a natural product tablet core to provide a coated tablet that consists almost entirely of Certified Organic ingredients and optional organic compliant ingredients, sugar coating has several major drawbacks. First, sugar coating techniques are often protracted and tedious multiple-step processes requiring skilled workers. Additionally, a relatively thick sugar coating is needed to produce a protective coating that is not susceptible to significant damage during handling. Such coating undesirably adds significantly to the caloric value of the finished coated tablet. Further, sugar coating processes generally involve a sealing step in which a polymer film, such as one comprised of polyvinylpyrrolidone, is coated onto the tablet core to strengthen the tablet core and impart better product stability. Additionally, sugar coating processes typically require, after the sealing step, a subcoating step, a smoothing step, and a polishing step. Because of the various difficulties associated with sugar coating, it is becoming an increasingly less popular tablet coating, with the majority of tablet coatings being polymer film coatings that are typically applied continuously to a moving bed of tablet cores, usually employing a spray technique.

Film coatings are typically comprised of a synthetic polymer, a cellulose derivative, or other film-forming polymer that cannot be certified as an organic ingredient, and which cannot be designated as an organic compliant ingredient. Examples include polyvinylpyrrolidone, polyvinyl acetate, methylcellulose, ethylcellulose, hydroxypropyl methylcellulose, hydroxypropylcellulose, and certain acrylic polymers, such as methacrylate and methylmethacrylate copolymers, and certain other vinyl polymers, such as polyvinyl alcohol. In addition, the conventional film-coating compositions incorporate one or more plasticizers that are generally selected from materials that are not Certified Organic materials and which cannot be designated as organic compliant materials, such as polyethylene glycols, triacetin, acetylated monoglyceride, citrate esters such as triethyl citrate and phthalate esters, such as diethyl phthalate. Such compositions also often require suspension stabilizers such as emulsifiers and thickeners and/or other additives that are not Certifiable Organic ingredients or capable of being designated as organic compliant. Accordingly, conventional polymer film coatings are not suitable for coating an all-natural compressed tablet core to provide a coated tablet that is to be designated as being comprised entirely of Certified Organic ingredients and optional organic compliant ingredients.

It is believed that known coating systems, and especially those applied using the preferred film coating techniques require synthetic and/or highly processed materials that cannot be Certified Organic ingredients and which cannot be designated as organic compliant. Further, it is believed that there are not any known equivalent all-natural compositions comprising only Certified Organic ingredients and optional organic compliant ingredients which exhibit suitable processing characteristics, such as suitable rheology, anti-adhesive properties, uniform coating thickness, drying times, etc., and which provide a suitable dried film coating having an acceptable appearance and mechanical properties.

### SUMMARY OF THE INVENTION

Conventional tablet coating compositions cannot be used for making coated tablets comprised entirely of Certified Organic ingredients and optional organic compliant ingredients. In one aspect of the invention there is provided a tablet coating composition that is comprised entirely of Certified Organic ingredients and optional organic compliant ingredients. Such coating composition of the invention may be applied to a tablet core that is comprised entirely of Certified Organic ingredients and optional organic compliant ingredients using conventional film-forming techniques to provide a finished coated tablet in which the coating is comprised entirely of Certified Organic ingredients and optional organic compliant ingredients, and wherein the finished coated tablet is comprised of about 95 percent or more Certified Organic ingredients by weight.

Various aspects of the invention have been achieved utilizing a tablet coating composition comprised primarily of Certified Organic honey solids and an organic compliant carrageenan that are dispersed in water.

These and other features, advantages and objects of the present invention will be further understood and appreciated by those skilled in the art by reference to the following specification and claims.

### DETAILED DESCRIPTION OF PREFERRED EMBODIMENTS

The coated tablets and tablet coating compositions of this invention are made from Certified Organic ingredients and organic compliant ingredients. The coating composition can be formulated for use in spraying or other conventional film-forming techniques to form a finished tablet coating made primarily or entirely of Certified Organic ingredients and organic compliant ingredients, and which exhibits excellent aesthetic and functional properties. In particular, the coatings of this invention may be applied using preferred economical and efficient spray coating techniques to provide an all-natural coating comprised of relatively inexpensive ingredients, and which may have a desirable smooth texture and shiny appearance, while providing mechanical and chemical properties that protect the tablet core against damage and degradation.

As used herein, the term “Certified Organic ingredients” means ingredients that are certified “organic” by the U.S.D.A. Accredited Certifying Agents for the National Organic Program established in accordance with the Organic Foods Production Act of 1990 (7 U.S.C. 6501 et seq.).

Generally, “Certified Organic products” are products that do not contain any artificial flavoring, coloring or chemical preservative (as defined by 21 CFR 01.22) or any other artificial or synthetic ingredient. Additionally, Certified Organic products are products that generally are not subjected to processing or are only subjected to minimal processing. Minimal processing may include traditional processes used to make a product edible or to preserve it or to make it safe for human consumption (e.g., smoking, roasting, freezing, drying or fermenting), or other physical processes which do not fundamentally alter the raw product and/or which only separate a whole intact food into component parts (e.g., grinding, separating, pressing).

Excluded from the category of “Certified Organic products” are most of the commonly employed tablet excipients including microcrystalline cellulose, cellulose derivatives, starch derivatives, polyvinylpyrrolidone, and other synthetic or highly processed lubricants and disintegrants. Also excluded from the category of “Certified Organic products” are most of the plasticizers, viscosity modifiers, synthetic or derivitized film-forming polymers and other ingredients commonly employed in tablet coating compositions.

As used herein, the expression “all-natural” refers to a composition that is comprised entirely of Certified Organic ingredients and optional organic compliant ingredients.

The term “organic compliant” refers to non-agricultural substances (as listed under 7 U.S.C. §205.605) that may be used in the production of products that may be labeled organic pursuant to 21 C.F.R. 01.22.

Typical sugar coatings have a relatively high moisture content and are easily damaged unless they are protected with a second coat exhibiting enhanced toughness, such as a shellac, polyvinylpyrrolidone or polyvinyl acetate film coating. Such coating systems do not provide a coating comprised primarily or entirely of Certified Organic ingredients and optional organic compliant ingredients. The coating systems described herein are comprised primarily or entirely of organic ingredients and optional organic compliant ingredients, and may be applied in a single coating step to achieve a continuous barrier on the tablet, which maintains the integrity of the tablet throughout the manufacturing, distribution and shelf-life of the product, and which protects the tablet core from physical damage due to abrasion and/or attrition.

In accordance various aspects of the invention, the tablet coating composition requires at least one Certified Organic carbohydrate that is capable of being dispersed in water to provide a coating composition that is capable of rapidly drying and forming a continuous film layer that surrounds a tablet core. Examples of carbohydrates include organic sugars, organic hydrocolloids, organic inulin, organic honey and organic honey solids.

A tablet coating composition in accordance with a preferred embodiment of the invention comprises water, Certified Organic honey solids, and an organic compliant carrageenan.

Honey solids or honey powder is composed of sugars (predominantly fructose and glucose, but may also include maltose and sucrose), dextrins, minerals, diastase enzyme (which converts starch to maltose units), small amounts of lipids and proteins other than the diastase enzyme, and beeswax.

The organic compliant carrageenan may be a κ-carrageenan, a λ-carrageenan or an τ-carrageenan. Also, combinations of κ-carrageenan, τ-carrageenan and λ-carrageenan may be employed. A suitable and currently preferred organic compliant carrageenan is λ-carrageenan.

It has been found that by adding a suitable quantity of organic honey solids and carrageenan to water, a film-forming, sprayable composition having good processing characteristics and which forms a suitable film coating upon drying can be provided.

It has been found desirable to add a small quantity of an organic compliant vegetable glycerin to certain tablet coating compositions of the invention in order to adjust the viscosity of the coating composition, such as to improve flowability and/or sprayability. Alternatively, or additionally, it has been found desirable to include the small amount of organic compliant vegetable glycerin as a plasticizer that functions to make the coating more flexible, and therefore tougher and more robust.

It has also been found desirable to add a small amount of Certified Organic soy lecithin to certain tablet coating compositions in accordance with the invention in order to reduce the tackiness of the coating on the tablet cores to prevent or reduce the possibility of tablet-to-tablet sticking during and after the coating process. Certified Organic soy lecithin has been found to function as an excellent anti-adhesion agent in the coating composition of the invention, and to act as an emulsifier for certain components in the honey solids providing improved dispersion of the honey solids and homogeneity of the composition. In addition, it is believed that the Certified Organic soy lecithin may function as a plasticizer or auxiliary plasticizer, and may enhance the film-forming properties of the selected carbohydrate (e.g., Certified Organic honey solids and carrageenan).

In the case of a tablet coating composition comprising a combination of Certified Organic honey solids and organic compliant carrageenan, a suitable amount of Certified Organic honey solids is in the range of from about 8 grams to about 15 grams per 100 grams of water. Lower amounts of honey solids tend to produce a coating that is difficult to dry, and higher amounts tend to produce a coating having an undesirably high viscosity that makes the coating process difficult. A suitable amount of organic compliant carrageenan in such composition is from about 0.6 grams to about 1 gram per 100 grams of water. Lower amounts of carrageenan tend to produce coatings that are difficult to dry, and higher amounts tend to produce a coating having an undesirably high viscosity that makes the coating process difficult. The organic compliant vegetable glycerin may be employed in such composition in an amount of from about 3.5 percent to about 5 percent of the total weight of the Certified Organic honey solids and the organic compliant carrageenan. Lower amounts of glycerin do not impart adequately improved flowability and/or sprayability, and higher amounts tend to undesirably reduce the total amount of Certified Organic ingredients, without a commensurate benefit. The Certified Organic soy lecithin may be employed in such composition in an amount of about 2.5 percent of the total weight of the Certified Organic honey solids and the organic compliant carrageenan. Appreciably higher amounts of lecithin could undesirably increase the viscosity of the coating, and appreciably lower amounts may not provide the desired and anti-adhesive, emulsifying, and/or plasticizing properties.

Other additives, such as colorants and/or flavorants, may be included in the tablet coating compositions of this invention. However, such additives are not generally desirable and are not typically included in all-natural products.

The all-natural coating compositions of this invention are intended primarily for use as coatings for all-natural or substantially all-natural tablet cores containing a dietary supplement, and more particularly to such tablet cores comprising at least 95 percent Certified Organic ingredients by weight, with the remaining ingredients in the tablet core, if any, being comprised of organic compliant materials. It is believed that such tablets are not known in the art or literature, especially such tablets exhibiting suitable hardness, friability and release of the active dietary supplement. However, U.S. Provisional Application No. 61/026,301 (Attorney Docket No. ENZ01 PP-300), which is hereby incorporated by reference, discloses all-natural or substantially all-natural dietary supplements in tablet form that may be used as tablet cores which can be coated with the compositions of this invention to provide a nutraceutically elegant coated tablet dosage form that is robust, easy to swallow, exhibits good shelf-life, and has desirable aesthetic properties. Desirably, such coated tablets will comprise about 95 percent or more Certified Organic ingredients by weight, with the balance, if any, of ingredients comprising organic compliant ingredients.

Because the coatings of this invention are relatively thin, typically ranging from a few micrometers to over 100 micrometers (e.g., 5 to 150 micrometers), and are typically comprised of at least about 90 percent Certified Organic ingredients, the resulting coated tablets typically comprise at least about 95 percent Certified Organic ingredients when the tablet core contains at least about 95 percent Certified Organic ingredients.

The coating compositions in accordance with this invention may be prepared by blending, mixing or dispersing the one or more Certified Organic carbohydrates and optional organic compliant ingredients in water, using generally any of various well-known mixing or blending apparatuses. Tablet cores can be coated with the tablet coating compositions of this invention using any of various known coating techniques and apparatuses, including rotating pan, fluid bed, spouted bed, etc.

The above description is considered that of the preferred embodiments only. Modifications of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A tablet coating composition comprising:
water; and
at least one Certified Organic ingredient dispersed in the water, the composition being comprised entirely of water, Certified Organic ingredients, and optional organic compliant ingredients.

2. A tablet coating composition comprising:
water;
Certified Organic honey solids; and
an organic compliant carrageenan.

3. A tablet coating composition as in claim 1, further comprising an organic compliant vegetable glycerin.

4. A tablet coating composition as in claim 1, further comprising a Certified Organic soy lecithin.

5. A tablet coating composition as in claim 1, further comprising an organic compliant vegetable glycerin and a Certified Organic soy lecithin.

6. A tablet coating composition as in claim 1, comprising from about 8 to about 15 grams of Certified Organic honey solids per 100 grams of water.

7. A tablet coating composition as in claim 1, comprising from about 8 grams to about 15 grams of Certified Organic honey solids, and from about 0.6 grams to about 1.0 grams of organic compliant carrageenan per 100 grams of water.

8. A tablet coating composition as in claim 7, further comprising an organic compliant vegetable glycerin in an amount of from about 3.5 percent to about 5 percent of the total weight of the Certified Organic honey solids and the organic compliant carrageenan.

9. A tablet coating composition as in claim 7, further comprising a Certified Organic soy lecithin in an amount of about 2.5 percent of the total weight of the Certified Organic honey solids and the organic compliant carrageenan.

10. A tablet coating composition as in claim 7, further comprising an organic compliant vegetable glycerin in an amount of from about 3.5 percent to about 5 percent of the total weight of the Certified Organic honey solids and the organic compliant carrageenan, and a Certified Organic soy lecithin in an amount of about 2.5 percent of the total weight of the Certified Organic honey solids and the organic compliant carrageenan.

11. A coated tablet, comprising:
a tablet core; and
a coating layer on the surface of the tablet core, the coating layer comprised entirely of Certified Organic ingredients and optional organic compliant ingredients.

12. A coated tablet as in claim 11, wherein the tablet core is comprised entirely of Certified Organic ingredients and optional organic compliant ingredients, the coated tablet being comprised of about 95 percent or more Certified Organic ingredients by weight.

13. A coated tablet as in claim 11, wherein the coating comprises Certified Organic honey solids and an organic compliant carrageenan.

14. A coated tablet as in claim 13, further comprising an organic compliant vegetable glycerin.

15. A coated tablet as in claim 13, further comprising a Certified Organic soy lecithin.

16. A coated tablet as in claim 13, further comprising an organic compliant vegetable glycerin and a Certified Organic soy lecithin.

17. A coated tablet as in claim 13, comprising from about 8 to about 15 grams of Certified Organic honey solids per 100 grams of water.

18. A coated tablet as in claim 13, comprising from about 8 grams to about 15 grams of Certified Organic honey solids, and from about 0.6 grams to about 1.0 grams of organic compliant carrageenan per 100 grams of water.

19. A coated tablet as in claim 18, further comprising an organic compliant vegetable glycerin in an amount of from about 3.5 percent to about 5 percent of the total weight of the Certified Organic honey solids and the organic compliant carrageenan.

20. A coated tablet as in claim 18, further comprising a Certified Organic soy lecithin in an amount of about 2.5 percent of the total weight of the Certified Organic honey solids and the organic compliant carrageenan.

21. A method of making a coated tablet comprised entirely of Certified Organic ingredients and optional organic compliant ingredients, comprising:
producing a tablet core comprised entirely of Certified Organic ingredients and optional organic compliant ingredients; and
coating the tablet core with a coating composition comprising at least one Certified Organic ingredient dispersed in water, the coating composition being comprised entirely of water, Certified Organic ingredients, and optional organic compliant ingredients.

22. The method of claim 21, wherein the coating comprises Certified Organic honey solids and an organic compliant carrageenan.

23. The method of claim 22, further comprising an organic compliant vegetable glycerin.

24. The method of claim 22, further comprising a Certified Organic soy lecithin.

25. The method of claim 22, further comprising an organic compliant vegetable glycerin and a Certified Organic soy lecithin.

26. A coated tablet comprising a tablet core and a coating layer on the surface of the tablet core, the coating layer comprised of honey solids and an organic compliant carrageenan.
